(19) United States
(12) Patent Application Publication
(10) Pub. No.: US 2009/0325856 A1
(43) Pub. Date: Dec. 31, 2009

(54) Composition Designed For The Treatment Of Multiple Sclerosis

(75) Inventor: Michel Geffard, Talence (FR)

Correspondence Address: YOUNG & THOMPSON, 209 Madison Street, Suite 500, Alexandria, VA, 22314, US

(73) Assignee: GEMAC, Cenon (FR)

(21) Appl. No.: 11/915,532

(22) PCT Filed: May 24, 2006
(86) PCT No.: PCT/FR2006/001217

§ 371 Date: May 8, 2008

(30) Foreign Application Priority Data
May 27, 2005 (FR) 0551400

(51) Int. Cl. A61K 38/00

(52) U.S. Cl. 514/2

(57) ABSTRACT

A composition for controlling the progression of multiple sclerosis includes at least:
One conjugate between poly-lysine and at least one fatty acid, and
One conjugate between poly-lysine and at least one antioxidant, and
One conjugate between poly-lysine and at least one amino acid derivative.

This invention relates to a composition for controlling the development of multiple sclerosis, comprising endogenic molecules grafted to poly-lysine, also called poly-lysine conjugates.

The invention also relates to the use of this composition.

Multiple sclerosis is an autoimmune, chronic, progressive disease that causes significant handicaps. Among the pathogenic processes, inflammation and activation of the immune system lead to the destruction of the myelin, a sheath that surrounds the nerve and participates in conducting the nerve impulse.

This inflammatory demyelination is reflected by the appearance of lesions distributed singularly on multiple plaques, dispersed in the central nervous system and able to affect any cerebral structure.

In the majority of patients suffering from multiple sclerosis, the disease first takes a so-called remittent form, then progresses gradually to result in handicaps, which, even if they do not affect life expectancy, significantly alter the quality of life. The process is then referred to as the secondary progressive phase. There are also progressive forms from the outset, called primary progressive forms, which are often more severe.

To evaluate the handicap level of patients suffering from multiple sclerosis, there is a scale called EDSS (Expanded Disability Status Scale), which refers to the functions involved. This scale is rated from 0 to 10, 0 corresponding to a normal examination, i.e., no handicap, and 10 corresponding to death owing to MS.

The remittent form of the disease is characterized by phases of relapse and remission. The relapses correspond to attacks of myelin and are marked by severe inflammation. The remission periods are due to the repair and the reorganization of the myelin and are reflected by a partial or total return to the clinical level corresponding to a level that is at least less than 3-4 of the EDSS scale, usually.

In the so-called progressive form of the disease, the periods of worsening are the result of an ineffective repair of the myelin and the appearance of scleroses, irreversible lesions. The handicap level on the EDSS scale increases and reaches the level 7; the patient essentially travels in a wheelchair.

Currently, there is no treatment that can arrest the progression of multiple sclerosis. Nevertheless, medications that can relieve patients by reducing the intensity and the frequency of handicapping relapses are used.

This is the case in particular of anti-inflammatory agents, in general corticoids that, administered at high doses, reduce the duration of the relapses.

There are also other medications that act as immunomodulators or immunosuppressors. The immunomodulators are conventionally used for the remittent forms. In particular, the beta interferons that reduce the number and the intensity of the relapses are known. For the advanced forms, immunosuppressors that slightly slow down the downward slide of the disease are used instead.

All of these medications act on a small part of the mechanisms responsible for demyelination but do not make it possible to treat it and even less to stop it. In addition, their administration generally requires the intervention of professionals and necessitates hospitalization and a special follow-up, which is burdensome and expensive for the patients. In contrast, these medications are all fraught with side-effects that greatly limit their prescription, in particular over the long term. They are therefore unsuitable for long-term treatment of a chronic disease such as multiple sclerosis.

Thus, there is a need for a treatment of multiple sclerosis, able to control the progression of the disease, easy to administer and able to be used over the long term.

This is to what this invention responds by proposing a composition for controlling the progression of multiple sclerosis, comprising at least:
One conjugate between poly-lysine and at least one fatty acid, and
One conjugate between poly-lysine and at least one antioxidant, and
One conjugate between poly-lysine and at least one amino acid derivative.

Antioxidants are defined as the known antioxidants and free radical scavengers.

Such a composition makes it possible to interrupt the destruction of the myelin, to promote the remyelination of the affected zones, to eliminate inflammation and to prevent the formation of new plaques. Consequently, it stops the progression of the disease, and if the handicap level reached is not too high, it also allows recovery and a reduction of the score on the EDSS scale.

It is known that multiple sclerosis is an autoimmune disease that is triggered by a causal agent that is unknown to date in individuals having genetic predispositions.

It begins by the entry into the body of virus-type or bacteria-type exogenic factors, for example, that activate the immune system and cause the opening or the physical rupture of the hematoencephalic barrier.

The opening of this barrier, which normally filters the passage and the diffusion in the central nervous system of any substance that circulates in the blood, therefore allows the passage of molecules in the central nervous system and in particular molecules and cells of the immune system that are activated by the external factors. There follows a spate of activations induced by the cells and the molecules that lead to the destruction of the myelin sheaths and the oligodendrocytes, myelin-producing cells.

One objective of the invention is therefore to prevent the induction of the processes that have taken place in the central nervous system by inhibiting the triggering of the immune and autoimmune responses induced by the exogenic factors and by blocking the hematoencephalic barrier.

For this purpose, the composition according to the invention contains in particular fatty acids that are grafted to poly-lysine, able to be recognized by the same cells and molecules of the immune system as those that are linked to the bacteria and viruses that are responsible for the disease and/or the chronicity, without, however, producing the immune response that is normally triggered by these same bacteria and viruses. Thus, the fatty acids of the composition according to the invention compete with the exogenic factors and cause a reduction of the response of the immune system. The fatty acids that are grafted to the poly-lysine have a decoy effect.

Furthermore, the fatty acids that are grafted to the poly-lysine and that are contained in the composition according to the invention prevent the activated cells of the immune system from passing through the hematoencephalic barrier and entering the central nervous system.

In addition to the direct destruction of the myelin, the activation of the immune system by the external factors causes in parallel the release, in excess, of reactive oxidized radicals (ROS) that are responsible for modifications of endogenic compounds that are then called neoantigens. These free radicals show direct cytotoxic effects and also alter the endogenic proteins, which increases the autoimmunity. Among these ROS, it is possible to cite in particular the nitrogen monoxide (NO) whose production is also increased by the lipopolysaccharides that are present on the surface of the disease-inducing external factors, in particular gram-negative bacteria. The NO would be involved in the opening of the hematoencephalic barrier and would also have a harmful action on the oligodendrocytes, thus preventing the formation of new myelin.

Another objective of this invention is therefore to monitor the induced oxidative processes so as to reduce the autoimmunity, to inhibit the opening of the hematoencephalic barrier and to reduce the cytotoxic effects on the central nervous system. Such monitoring would also make it possible to restore functional oligodendrocytes, myelin-producing cells.

Actually, the composition according to the invention contains antioxidants that are conjugated with the poly-lysine that scavenge the oxidized radicals and inhibit the pathogenic oxidative processes. By scavenging the reactive oxidized radicals and the free radicals, the composition according to the invention makes possible in particular neuronal and myelinic protection as well as a reduction of the inflammation.

To control the progression of multiple sclerosis even more effectively, it is also suitable that the administered treatment have a neurotrophic action. Thus, the composition according to the invention contains amino acid derivatives that are grafted to the poly-lysine and that are able to ensure a neuronal protection in particular by strengthening the membrane adhesion of the myelin and the axon.

In addition, these amino acid derivatives that are grafted to the poly-lysine also have an immunomodulating effect and make it possible to control the autoimmune responses.

Finally, whereby one of the primary characteristics of multiple sclerosis is inflammation, the composition according to the invention also has a direct anti-inflammatory action, in particular due to the presence of fatty acids that are grafted to the poly-lysine.

This shows the synergetic effects of the composition according to the invention.

The composition according to this invention therefore makes it possible to control the progression of multiple sclerosis, both:
By reducing the immune response that is induced by the exogenic factors, owing to the presence of fatty acids that can compete with the latter on the level of specific molecules and cells of the immune system,
By reinforcing the hematoencephalic barrier, owing to the presence of fatty acids and antioxidants, whereby the latter are capable of scavenging the NO that is involved in the opening of the barrier and the formation of neoantigens,
By scavenging the reactive oxidized radicals that are responsible for much damage, owing to the presence of antioxidants, which makes possible in particular the myelenic repair by stimulation of the precursor oligodendrocytes,
By ensuring neuronal protection by amino acid derivatives,
By regulating the autoimmune responses, owing to the presence of amino acid derivatives, and
By directly reducing the inflammation owing to the presence of fatty acids that have an anti-inflammatory action.

Thus, the three types of endogenic molecules that are contained in the composition according to the invention have complementary and potentializing actions that make it possible to affect the different aspects of multiple sclerosis. Even so, to be effective, these molecules cannot be used in unbonded form because they would be quickly metabolized, would not reach their target and would not have any therapeutic action.

Actually, according to the invention, these endogenic molecules are grafted to a particular vector: the poly-lysine. This particular vector makes it possible:
To prevent the metabolic degradation of the endogenic molecules,
To allow the passage of endogenic molecules through the hematoencephalic barrier,
To reach their targets with endogenic molecules, and
To provide to the endogenic molecules a therapeutic action that they do not have without this grafting, in particular for fatty acids.

Furthermore, the poly-lysine also has the advantage of being inert, hypoallergenic, non-immunogenic and of having an adequately long half-life.

Advantageously, the composition according to the invention contains only endogenic substances, i.e., substances that are known to be present naturally in the living. It does not have any toxicity, nor secondary effects, and can be administered over the long term.

The composition according to the invention can be incorporated in different types of pharmaceutical preparations that are presented in all galenical forms. Among the galenical forms of the pharmaceutical preparations that can include the active ingredient according to the invention, it is possible to cite in particular the sublingual form, practical for use and equivalent in effectiveness to a subcutaneous administration.

Other characteristics and advantages will emerge from the description of the composition examples according to the invention that will follow, non-limiting, opposite the accompanying drawings in which the different figures represent:

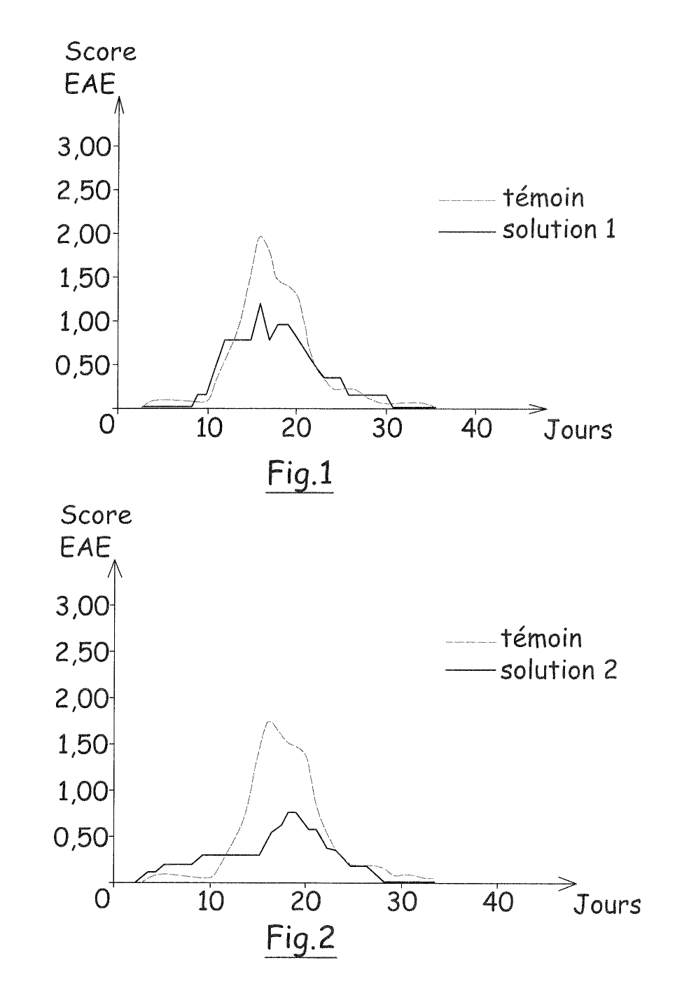

FIG. 1, a graphic representation of the effect of a sodium chloride solution (NaCl) on an EAE (experimental allergic encephalitis) attack induced in female Lewis rats and the effect of a solution of fatty acids conjugated with poly-L-lysine (solution 1) on this same EAE attack,

FIG. 2, a graphic representation of the effect of an NaCl solution on an EAE attack induced in female Lewis rats and the effect of a solution of fatty acids conjugated with poly-L-lysine (solution 2) on this same EAE attack,

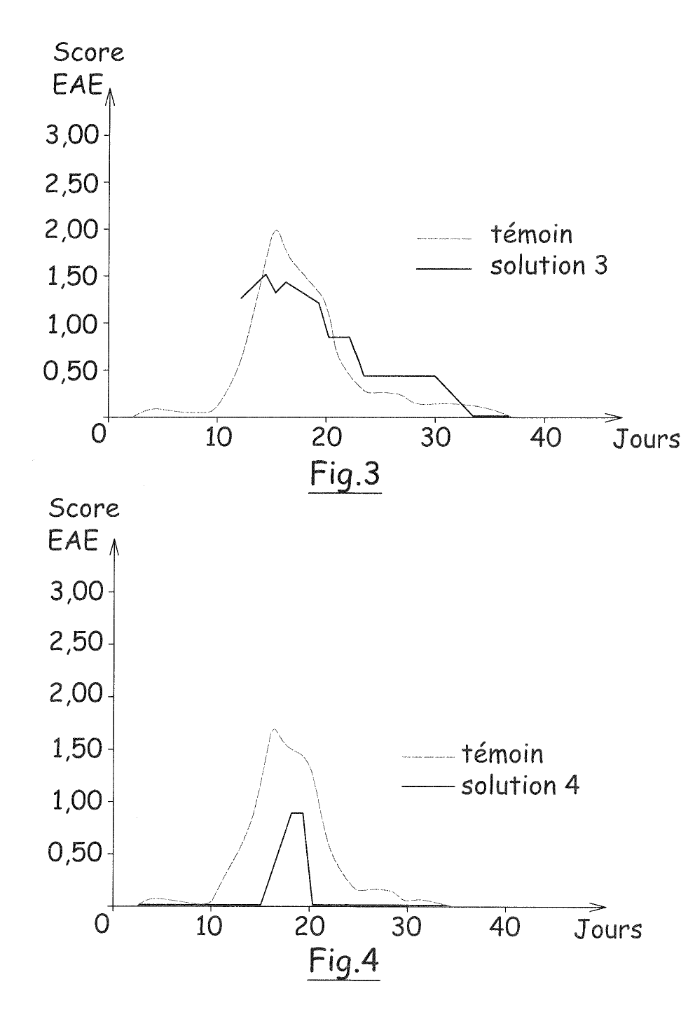

FIG. 3, a graphic representation of the effect of an NaCl solution on an EAE attack induced in female Lewis rats and the effect of a solution of antioxidants conjugated with poly-L-lysine (solution 3) on this same EAE attack,

FIG. 4, a graphic representation of the effect of an NaCl solution on an EAE attack induced in female Lewis rats and the effect of a solution of anti-oxidants conjugated with poly-L-lysine and fatty acids conjugated with poly-L-lysine (solution 4) on this same EAE attack,

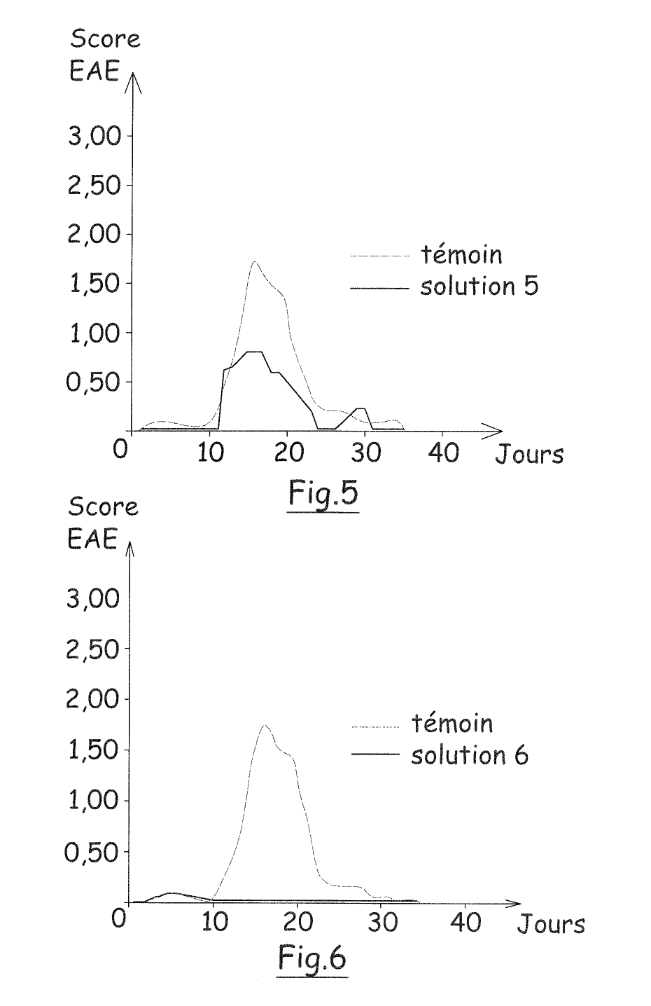

FIG. 5, a graphic representation of the effect of an NaCl solution on an EAE attack that is induced in rats and the effect of a solution of anti-oxidants that are conjugated with poly-L-lysine, fatty acids that are conjugated with poly-L-lysine and amino acids that are conjugated with poly-L-lysine (solution 5) on this same EAE attack,

FIG. 6, a graphic representation of the effect of an NaCl solution on an EAE attack that is induced in female Lewis rats and the effect of a solution of anti-oxidants that are conjugated with poly-L-lysine, fatty acids that are conjugated with poly-L-lysine and amino acids that are conjugated with poly-L-lysine (solution 6) on this same EAE attack,

FIG. 7, a graphic representation of the dose effect of the composition according to the invention on an acute EAE, and

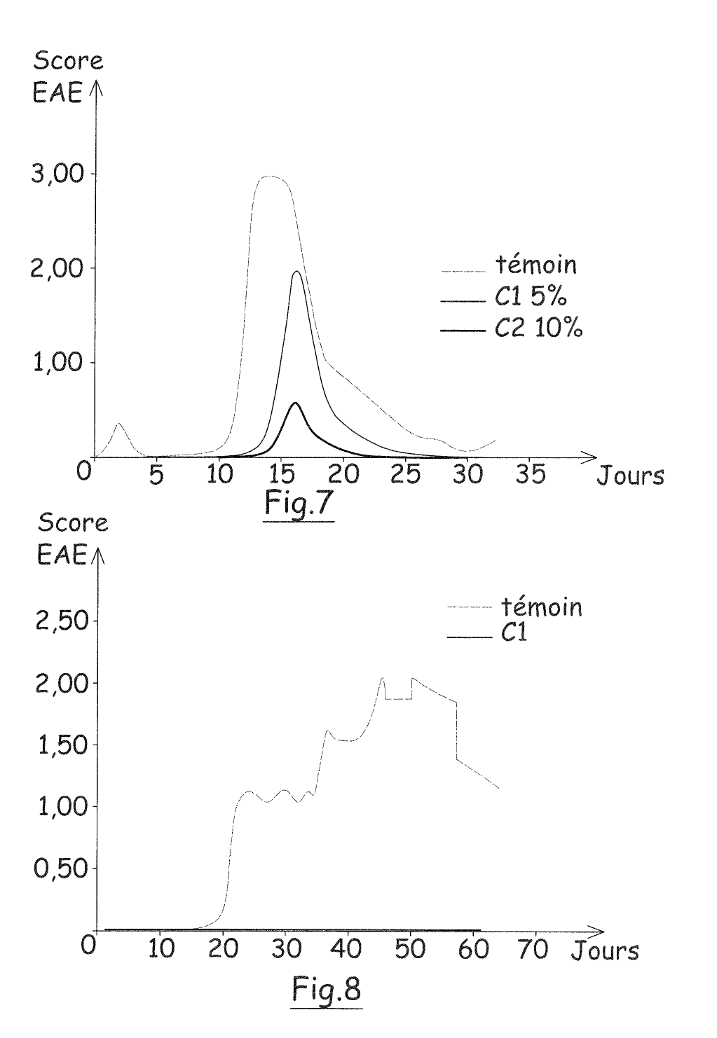

FIG. 8, a graphic representation of the effect of the composition according to the invention on a chronic EAE.

1. Example of Composition that is Suitable for the Secondary Progressive Form

Among the compositions according to the invention, it is possible to cite a composition that contains the following poly-L-lysine conjugates:
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine-glutaric anhydride-poly-L-lysine

Such a composition is particularly suitable for the treatment of the secondary progressive form of multiple sclerosis, and in particular for patients whose handicap is less than 7 on the EDSS scale.

This composition can be packaged in a single form (C1), comprising the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine—glutaric anhydride-poly-L-lysine
L-Histidine-glutaric anhydride-poly-L-lysine
5-Methoxytryptamine glutaric anhydride-poly-L-lysine

According to a variant, the composition of Example 1 can be packaged in two forms corresponding to two separate pharmaceutical preparations. This variant allows optimization of administration by taking the composition according to the invention twice.

According to this variant, the first form (C2) comprises the following components:

Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine

According to this variant, the second form (C3) comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic cid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine glutaric anhydride-poly-L-lysine

### 2. Example of Composition Suitable for the Remittent Form

Another composition example according to the invention contains the following poly-L-lysine conjugates:
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Oleic acid-poly-L-lysine-retinoic acid
Oleic acid-poly-L-lysine-co-enzyme Q10
Alpha-tocopherol succinate-poly-L-lysine
Ascorbic acid-poly-L-lysine
L-Methionine-reduced glutaraldehyde-poly-L-lysine
L-Cysteine-reduced glutaraldehyd-poly-L-lysine
Taurine-reduced glutaraldehyde-poly-L-lysine
5-Methoxytryptamine-glutaric anhydride-poly-L-lysine

Such a composition is particularly suitable for treatment of the remittent form of multiple sclerosis.

This composition can be packaged in a pharmaceutical preparation in a single form (C4) that comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Oleic acid-poly-L-lysine-retinoic acid
Oleic acid-poly-L-lysine-co-enzyme Q10
Alpha-tocopherol succinate-poly-L-lysine
Ascorbic acid-poly-L-lysine
L-Methionine reduced glutaraldehyde-poly-L-lysine
L-Cysteine-reduced glutaraldehyde-poly-L-lysine
Taurine-reduced glutaraldehyde-poly-L-lysine
5-Methoxytryptamine-glutaric anhydride-poly-L-lysine

### 3. Example of Composition Suitable for the Secondary Progressive Form and the Remittent Form

Among the compositions according to the invention, it is also possible to cite a composition (CS) that contains the following poly-L-lysine conjugates:
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Oleic acid-poly-L-lysine-retinoic acid
Oleic acid-poly-L-lysine-co-enzyme Q10
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
5-Methoxytryptamine-glutaric anhydride-poly-L-lysine
Histamine-glutaric anhydride-poly-L-lysine
Histidine-glutaric anhydride-poly-L-lysine.

Such a composition is suitable for treatment both of the remittent form and the secondary progressive form of multiple sclerosis.

This composition can be packaged in a pharmaceutical preparation in a single form or in several forms.

### 4. Evaluation of the Effectiveness and the Toxicity of the Composition According to the Invention

The experimental model used for these studies is experimental allergic encephalitis (EAE). It is an animal model whose attack mimics the relapse of multiple sclerosis.

4.1 Studies on an Acute EAE Animal Model

4.1.1 Effects of Endogenic Molecules Grafted to Poly-Lysine
This study has as its object to evaluate in the animal the complementary effects of different types of molecules conjugated with the poly-L-lysine that are present in the composition according to the invention.
The EAE model is carried out on 7-week-old female Lewis rats by injecting an emulsion of myelin basic protein and Mycobacterium Tuberculosis.
The clinical evaluation is carried out according to the following scale of neurological signs:
   0: No sign
   0.5: Low activity and loss of weight
   1: Hypotonia of the tail
   2: Weakness of the rear members
   3: Hemiparesis
   4: Tetraplegia, moribund state
The protocol consists in injecting 0.5 ml of the solution to be tested or NaCl (control) subcutaneously once per day for 30 days, 9 days after the induction of the EAE.
The compositions of the tested solutions are:
Solution 1:
   Oleic acid-poly-L-lysine,
   Palmitic acid-poly-L-lysine, and
   Myristic acid-poly-L-lysine.
Solution 2:
   Azelaic acid-poly-L-lysine,
   Myristic acid-poly-L-lysine,
   Palmitic acid-poly-L-lysine,
   Palmitoleic acid-poly-L-lysine, and
   Trans, trans-farnesyl-L-cysteine-poly-L-lysine.
Solution 3:
   L-Cysteine-glutaric anhydride-poly-L-lysine,
   Homocysteine-glutaric anhydride-poly-L-lysine,
   L-Methionine-glutaric anhydride-poly-L-lysine,
   Taurine-glutaric anhydride-poly-L-lysine,
   Alpha-Tocopherol-succinate-poly-L-lysine,
   L-Cysteine-glutaraldehdye-poly-L-lysine,
   Homocysteine-glutaraldehyde-poly-L-lysine,
   L-Methionine-glutaraldehyde-poly-L-lysine,
   Taurine-glutaraldehyde-poly-L-lysine, and
   Vitamin C-poly-L-lysine.
Solution 4:
   Azelaic acid-poly-L-lysine,
   Palmitoleic acid-poly-L-lysine,
   Trans, trans-farnesyl-L-cysteine-poly-L-lysine,
   Oleic acid-poly-L-lysine,
   Palmitic acid-poly-L-lysine,
   Myristic acid-poly-L-lysine,
   L-Cysteine-glutaric anhydride-poly-L-lysine,
   Alpha-tocopherol succinate-poly-L-lysine,
   Taurine glutaraldehyde-poly-L-lysine, and
   Vitamin C-poly-L-lysine.
Solution 5:
   Azelaic acid-poly-L-lysine,
   Palmitoleic acid-poly-L-lysine,
   Trans, trans-farnesyl-L-cysteine-poly-L-lysine,
   Oleic acid-poly-L-lysine,
   Palmitic acid-poly-L-lysine,
   Myristic acid-poly-L-lysine,
   Retinoic acid-poly-L-lysine,
   L-Methionine-glutaric anhydride-poly-L-lysine,
   Taurine-glutaric anhydride-poly-L-lysine,
   Alpha-tocopherol succinate-poly-L-lysine,
   L-Cysteine-glutaraldehyde-poly-L-lysine,
   L-Methionine-poly-L-lysine,
   Taurine-poly-L-lysine,
   Vitamin C-poly-L-lysine,
   Tryptophan-glutaric anhydride-poly-L-lysine,
   Cholesterol-glutaric anhydride-poly-L-lysine,
   Tryptophan-glutaraldehyde-poly-L-lysine, and
   GABA-glutaraldehyde-poly-L-lysine.
Solution 6:
   Azelaic acid-poly-L-lysine,
   Palmitoleic acid-poly-L-lysine,
   Trans, trans-farnesyl-L-cysteine-poly-L-lysine,
   Oleic acid-poly-L-lysine,
   Palmitic acid-poly-L-lysine,
   Myristic acid-poly-L-lysine,
   Homocysteine-glutaraldehyde-poly-L-lysine,
   Taurine-glutaric anhydride-poly-L-lysine,
   Alpha-tocopherol succinate-poly-L-lysine,
   L-Cysteine-glutaraldehyde-poly-L-lysine,
   L-Methionine-glutaraldehyde-poly-L-lysine,
   Vitamin C-poly-L-lysine,
   Hydroxytryptophan-glutaraldehyde-poly-L-lysine,
   5-Methoxytryptamine-glutaraldehyde-poly-L-lysine, and
   Tyrosine-glutaraldehyde-poly-L-lysine.

The results that are recapitulated in FIGS. 1 to 6 show in a first step that fatty acids that are conjugated with poly-lysine alone do not have an effect that is adequate for controlling the attack.

On the contrary, it is noted that by adding antioxidants that are conjugated with poly-lysine to these fatty acids that are conjugated with poly-lysine, a beneficial effect is obtained.

Finally, if amino acids that are conjugated with poly-lysine are added to these fatty acids and antioxidants that are conjugated with poly-lysine, a complete inhibition of the attack is achieved.

Thus, the three types of compounds that are contained in the composition according to the invention act in synergy.

4.1.2 Evaluation of the Effect of the Composition According to the Invention on the Acute EAE
a—Evaluation of the Dose Effect of the Preparation C1 of Example 1
   The objective of this study is to see if the effect of the composition according to the invention is dependent upon the dose administered.
   The EAE model is carried out on 7-week-old female Lewis rats by injecting an emulsion of myelin basic protein and Mycobacterium Tuberculosis.
   The clinical evaluation is carried out according to the following scale of neurological signs:
      0: No sign
      0.5: Low activity and loss of weight
      1: Hypotonia of the tail
      2: Weakness of the rear members
      3: Hemiparesis
      4: Tetraplegia, moribund state
   The protocol consists in injecting one of the following treatments subcutaneously once per day, six out of seven days, for 30 days, beginning on the day of the induction of the EAE:
      0.5 ml of NaCl (control)
      5 mg/kg of C1 dosed at 1%,
      12.5 mg/kg of C1 dosed at 2.5%,
      25 mg/kg of C1 dosed at 5%, or
      50 mg/kg of C1 dosed at 10%.
   The results are presented in FIG. 7.
   It is noted that the larger the dose of C1 administered, the more the EAE attack is reduced.
b—Evaluation of the Dose Effect of the Preparation C5 of Example 3
   The EAE model is carried out on 7-week-old female Lewis rats by injecting an emulsion of myelin basic protein and Mycobacterium Tuberculosis.
   The clinical evaluation is carried out according to the following scale of neurological signs:
      0: No sign
      0.5: Low activity and loss of weight
      1: Hypotonia of the tail
      2: Weakness of the rear members
      3: Hemiparesis
      4: Tetraplegia, moribund state
   The protocol consists in injecting one of the following treatments subcutaneously once per day, six out of seven days, for 25 days, beginning 8 days after the induction of the EAE:
      0.5 ml of NaCl (control),
      4 mg/kg of C5 dosed at 1%,
      10 mg/kg of C5 dosed at 2.5%,
      12 mg/kg of C5 dosed at 5%,
      30 mg/kg of C5 dosed at 7.5%, or
      40 mg/kg of C5 dosed at 10%.
   The results that are obtained also show that the greater the dose of C5 that is administered, the more the EAE attack is reduced.
c—Evaluation of the Effect of the Composition According to the Invention on the Infiltration of Leukocytes in the Brain
   The objective of this study is to see if the composition according to the invention helps in the reduction of the permeability of the hematoencephalic barrier by enumerating by immunocytochemistry the structures of the central nervous system (CNS) that are infiltrated by the leukocytes that are activated after induction of an EAE.
   The EAE model is carried out on female Lewis rats by injecting an emulsion of myelin basic protein and Mycobacterium tuberculosis.
   The clinical evaluation if carried out under the following scale of neurological signs:
      0: No sign
      0.5: Low activity and loss of weight
      1: Hypotonia of the tail
      2: Weakness of the rear members
      3: Hemiparesis
      4: Tetraplegia, moribund state
   The protocol consists in injecting the solution to be tested or the NaCl (control) subcutaneously once per day for 30 days, 9 days after the induction of the EAE.
   The tested solutions are:
      Solution 1: the preparation C1 of Example 1
      Solution 2: the preparation C2 of Example 1
      Solution 3: the preparation C3 of Example 1
   The immunocytochemical tests are done using the anti-CD 45 antibody on sections of rat brains fixed in paraformaldehyde.
   The immunocytochemical analysis makes it possible to show the number of infiltrated cerebral structures. The results are presented in the following table:

| | NaCl | C1 | C2 | C3 |
|---|---|---|---|---|
| Number of Infiltrated SNC Structures | 28 | 5 | 6 | 5 |

These results show that the composition according to the invention greatly reduces the leukocytic infiltration in the central nervous system.
The composition according to the invention therefore reduces the permeability of the hematoencephalic barrier and plays an immunomodulatory role.

### 4.2 Studies on the Chronic EAE Animal Model

4.2.1. Evaluation of the Effect of the Composition According to the Invention on the EAE Attack
The EAE model is carried out on 10- to 11-week-old female Lewis rats by injecting an emulsion of myelin oligodendrocyte glycoprotein and Mycobacterium Tuberculosis.
The clinical evaluation is carried out according to the following scale of neurological signs:
   0: No sign
   0.5: Low activity and loss of weight
   1: Hypotonia of the tail
   2: Weakness of the rear members
   3: Hemiparesis
   4: Tetraplegia, moribund state
The protocol consists in injecting one of the following treatments subcutaneously:
   3.25 mg of C1 dosed at 10% twice per day, six out of seven days, for 60 days, beginning 8 days after the induction of the EAE, or
   0.25 ml of NaCl twice per day, six out of seven days, for 55 days, beginning 8 days after the induction of the EAE.
The results that are obtained and are presented in FIG. 8 show that the composition according to the invention totally inhibits the EAE attack.

4.2.2 Evaluation of the Effect of the Composition on the Neuroprotective Action
The objective of this study is to see if the composition according to the invention has a neuroprotective effect.
Immunocytochemistry techniques are used in the tissues of the central nervous system (CNS).
Three effects are evaluated:
   The protection of the myelin
   The passage of the hematoencephalic barrier by the molecules that enter the composition according to the invention, and
   The general structure of the nervous tissue.
The EAE model is carried out on 10- to 11-week-old female Lewis rats by injecting an emulsion of myelin oligodendrocyte glycoprotein and Mycobacterium Tuberculosis.
The clinical evaluation is carried out according to the following scale of neurological signs:
   0: No sign
   0.5: Low activity and loss of weight
   1: Hypotonia of the tail
   2: Weakness of the rear members
   3: Hemiparesis
   4: Tetraplegia, moribund state
The protocol consists in injecting one of the following treatments subcutaneously:
   3.25 mg of C1 dosed at 10% two times per day, six out of seven days, for 60 days, beginning 8 days after the induction of the EAE, or
   0.25 ml of NaCl two times per day, six out of seven days, for 55 days, beginning 8 days after the induction of the EAE.
a—Evaluation of the Effect on Myelin
   The objective is to evaluate the effect of the composition according to the invention on myelin, insulting sheath that protects the axon and allows the nerve impulse to circulate more quickly.
   For this purpose, a marker of myelin, basic anti-protein antibody of myelin, is used, and the results are visualized by immunocytochemistry on sections of rat brains.
   The results show that the organization of the myelin is preserved in the treated rats, whereas the myelin is completely disorganized in the NaCl-treated rats (negative control).
   The composition according to the invention therefore has a protective effect of the myelin.
b—Evaluation of the Passage of the Hematoencephalic Barrier by the Molecules that Enter into the Composition According to the Invention
   The objective is to detect the presence of a therapeutic molecule of the composition according to the invention, the methionine that is linked to the poly-lysine, at the central nervous system.
   For this purpose, anti-methionine antibodies are used, and the results are visualized by immunocytochemistry on sections of rat brains.
   The results show:
      An immunoreactivity in the rats that are treated with the composition according to the invention, and
      No immunoreactivity in the rats that are treated with the NaCl (negative control).
   The presence of the methionine that is linked to the poly-L-lysine at the central nervous system of the treated rats shows that the molecule passes through the hematoencephalic barrier.
   These results therefore suggest that the therapeutic molecules that constitute the composition according to the invention could pass through the hematoencephalic barrier.
c—Evaluation of the Effect on the General Structure of the Nerve Tissue
   The objective is to evaluate the effect of the composition according to the invention on the state of the structure of the nerve tissue of rats.
   For this purpose, trophic anti-factor antibodies are used, whereby the loss of its factors is characteristic of a neuronal disorder, and the results are visualized by immunocytochemistry and by histology techniques on sections of rat brains.
   The results show:
      A disorganization of myelin in the bone marrow of rats that are treated with NaCl (negative control), and
      A normal organization of myelin in the bone marrow of rats that are treated with the composition according to the invention.
   The composition according to the invention therefore makes it possible to preserve the general structure of the nerve tissue.
   All of these results show that the composition according to the invention indeed has a neuroprotective effect.

### 4.3 Toxicity Studies

4.3.1 Study of Toxicity by One-Time Administration
This study consists in injecting the preparation C1, C2 or C3 of Example 1 according to the invention into rats intravenously with a single dose.
The clinical observation is made on the day of administration 30 minutes, 1 hour, 2 hours, 3 hours and 4 hours after the administration and then at least once per day for 14 days.
This clinical observation shows that the lethal dose 0 (LDO) and the lethal dose 50 (LD50) for C1, C2 and C3 are more than 10 ml/kg in rats (or 10 mg per kg of C1, C2 or C3).

4.3.2 Study of Acute Toxicity
To carry out this study, C2 and C3 of Example 1 according to the invention are administered to Sprague-Dawley rats by subcutaneous injection.
The animals receive a 2 ml injection of C2 or C3 three times per day the first day and then 1 ml for 3 to 7 days.
0% mortality is observed. All the animals survive at the injected doses and are in good general condition.
No change is observed either in weight gain or loss or in the behavior of the animals.

4.3.3 Study of Chronic Toxicity
The study is carried out on Sprague-Dawley rats, aged 4 to 8 weeks.
The treated rats are first treated in acute toxicity, and then receive a subcutaneous injection of 0.5 ml of C2 or C3 per day for 150 to 180 days.
The control rats receive 0.5 ml of NaCl for this same period.
The results that are obtained reveal that there is no change in weight gain or loss, nor any adverse effect. All of the animals are alive and have a homogeneous and uniform weight gain or loss that can be superposed on that of the controls.
Thus, the composition according to the invention does not exhibit any apparent toxicity.

4.3.4 Immunotoxicity
The objective of this study is to verify, by the production of antibodies against the poly-L-lysine conjugates, the presence or the absence of reaction that increases awareness of these compounds following the uniform and extended injection of solutions.
The production of antibodies is evaluated with the aid of ELISA immunological tests. In rat serums, the direct ELISA technique makes it possible to demonstrate the absence of antibodies directed against one of the conjugates that are optionally present in the solutions.
The study is carried out on 8-week-old male Sprague-Dawley rats. The tests are carried out for 6 months on:
   Rats that do not receive any injection (control rats),
   Rats that receive one injection of 0.5 ml per day, 5 days per week, of a solution that is close to preparation C2 of Example 1 according to the invention, and
   Rats that receive an injection of 0.5 ml per day, 5 days per week, of NaCl.
The results that are obtained for this study show that there is no significant immunological response against each of the compounds tested in the serums of rats that have received an injection of the solution that is close to C2 for 6 months.
The composition according to the invention therefore does not exhibit any immunotoxicity.

The various studies of toxicity carried out on the composition according to the invention show that it is non-toxic and that it can therefore be recommended for long-term chronic treatment.

Of course, the invention is obviously not limited to the examples of compositions shown and described above, but on the contrary covers all the variants, in particular regarding fatty acids, antioxidants, and the amino acid derivatives that are used, as well as the preparations that can include the composition.

1. Composition that is useful for the preparation of a medication that is designed to control the progression of multiple sclerosis, characterized in that it comprises at least:
One conjugate between poly-lysine and at least one fatty acid, and
One conjugate between poly-lysine and at least one antioxidant, and
One conjugate between poly-lysine and at least one amino acid derivative.

2. Composition according to claim 1, wherein it comprises the following poly-L-lysine conjugates:
Azelaic acid-poly-L-lysine-oleic acid
Azelaic acid-poly-L-lysine-palmitoleic acid
Trans, trans-farnesyl-L-cysteine-poly-L-lysine-13 oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine-glutaric anhydride-poly-L-lysine.

3. Composition according to claim 1, wherein it is packaged in a single form that comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine-glutaric anhydride-poly-L-lysine

4. Composition according to claim 1, wherein it is packaged in two forms, whereby the first form comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine palmitic acid
Oleic acid-poly-L-lysine myristic acid
Oleic acid-poly-L-lysine-linoleic acid
Oleic acid-poly-L-lysine thioctic acid
Cholesterol-poly-L-lysine-oleic acid
Linoleic acid-poly-L-lysine,
and the second form comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine-glutaric anhydride-poly-L-lysine.

5. Composition according to claim 1, wherein it comprises the following poly-L-lysine conjugates:
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Oleic acid-poly-L-lysine-retinoic acid
Oleic acid-poly-L-lysine-co-enzyme Q10
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
5-Methoxytryptamine-glutaric anhydride-poly-L-lysine
Histamine-glutaric anhydride-poly-L-lysine
Histidine-glutaric anhydride-poly-L-lysine.

6. Composition according to claim 2, wherein it is suitable for treatment of the secondary progressive form of multiple sclerosis.

7. Composition according to claim 1, wherein it comprises the following poly-L-lysine conjugates:
Azelaic acid-poly-L-lysine-oleic acid
Azelaic acid-poly-L-lysine palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Oleic acid-poly-L-lysine-retinoic acid
Oleic acid-poly-L-lysine-co-enzyme Q10
Alpha-tocopherol succinate-poly-L-lysine
Ascorbic acid-poly-L-lysine
L-Methionine-reduced glutaraldehyde-poly-L-lysine
L-Cysteine-reduced glutaraldehyde poly-L-lysine
Taurine-reduced glutaraldehyde-poly-L-lysine
5-Methoxytryptamine-glutaric anhydride-poly-L-lysine

8. Composition according to claim 5, wherein it is suitable for the treatment of the remittent form of multiple sclerosis.

9. A method for controlling the progression of multiple sclerosis comprising administering to a patient in need thereof an effective amount of a composition comprising:
One conjugate between poly-lysine and at least one fatty acid, and
One conjugate between poly-lysine and at least one antioxidant, and
One conjugate between poly-lysine and at least one amino acid derivative.

10. The method according to claim 9, wherein the composition reduces the immune and autoimmune responses by inhibiting the exogenic factors that are responsible for multiple sclerosis.

11. The method according to claim 9, wherein the composition reduces the inflammation by scavenging the reactive oxidized radicals and the free radicals.

12. The method according to claim 9, wherein the composition reduces the permeability of the hematoencephalic barrier, in particular by scavenging the nitrogen monoxide involved in the opening of said barrier.

13. The method according to claim 9, wherein the composition provides the neuronal and myelinic protection by scavenging the free radicals and by reinforcing the membrane adhesion of the myelin and the axon.

14. The method according to claim 9, wherein the composition provides the myelinic repair by stimulation of the precursor oligodendrocytes.

15. Composition according to claim 2, wherein it is packaged in two forms, whereby the first form comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine,
and the second form comprises the following components:
Ethyl alcohol 96%
Purified water
Azelaic acid-poly-L-lysine-oleic acid
Azelaic acid-poly-L-lysine-palmitoleic acid
Trans, trans-farnesyl-L-cysteine-poly-L-lysine-oleic acid
Oleic acid-poly-L-lysine-palmitic acid
Oleic acid-poly-L-lysine-myristic acid
Oleic acid-poly-L-lysine-linoleic acid
Oleic acid-poly-L-lysine-thioctic acid
Cholesterol-poly-L-lysine-oleic acid
Linoleic acid-poly-L-lysine
Oleic acid-poly-L-lysine-palmitoleic acid
Trans, trans-farnesyl-L-cysteine-poly-L-lysine-palmitic acid
Alpha-tocopherol succinate-poly-L-lysine
Ascorbic acid-poly-L-lysine
L-Methionine-reduced glutaraldehyde-poly-L-lysine
L-Cysteine-reduced glutaraldehyde-poly-L-lysine
Taurine-reduced glutaraldehyde-poly-L-lysine
Histamine-glutaric anhydride-poly-L-lysine
L-Histidine-glutaric anhydride-poly-L-lysine
5-Methoxytryptamine-glutaric anhydride-poly-L-lysine.

16. Composition according to claim 3, wherein it is suitable for treatment of the secondary progressive form of multiple sclerosis.

17. Composition according to claim 4, wherein it is suitable for treatment of the secondary progressive form of multiple sclerosis.

18. Composition according to claim 5, wherein it is suitable for treatment of the secondary progressive form of multiple sclerosis.

19. Composition according to claim 7, wherein it is suitable for the treatment of the remittent form of multiple sclerosis.
